(19) United States
(12) Patent Application Publication
(10) Pub. No.: US 2008/0113019 A1
(43) Pub. Date: May 15, 2008

(54) COMPOSITION FOR CAPSULE COATING

(75) Inventors: Hideo Satokawa, Kawasaki-shi (JP); Kiyonori Furuta, Kawasaki-shi (JP); Keiji Iwasaki, Kawasaki-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: AJINOMOTO CO., INC., Tokyo (JP)

(21) Appl. No.: 11/951,678

(22) Filed: Dec. 6, 2007

Related U.S. Application Data
(63) Continuation of application No. PCT/JP2006/311695, filed on Jun. 6, 2006.

(30) Foreign Application Priority Data
Jun. 6, 2005 (JP) 2005-165652

(51) Int. Cl. A61K 9/48

(52) U.S. Cl. 424/451

(57) ABSTRACT

The present invention provides a composition that is suitable for producing a plant-derived, practical capsule shell sheet having both sufficient strength and elongation as a capsule shell, superior adhesiveness and transparency, and a low burden on a drying step. The present invention also provides a capsule shell sheet obtained using the composition and a capsule preparation process. The composition of the present invention contains vegetable polysaccharides and a polyglutamic acid.

### CROSS-REFERENCE TO RELATED APPLICATIONS

The present application is a continuation of PCT/JP2006/311695, filed on Jun. 6, 2006, and claims priority to JP 2005-165652, filed on Jun. 6, 2005, the entire contents of these applications is incorporated herein by reference.

### BACKGROUND OF THE INVENTION

1. Field of the Invention

The present invention provides a composition for producing a capsule shell, which comprises (A) a vegetable polysaccharide and (B) polyglutamic acid (sometimes to be referred to as a composition for a capsule shell). The present invention further provides a composition for a capsule shell comprising (A) vegetable polysaccharide, (B) polyglutamic acid, and at least one additional ingredient selected from (C) water, (D) a plasticizer, (E) glycerin fatty acid ester and (F) oil. In addition, the present invention provides a capsule shell sheet and a capsule preparation.

2. Discussion of the Background

Capsule shells are generally produced using gelatin as a main component. However, in recent years, gelatin has fallen into disfavor amongst religious people and vegetarians because it is derived from animals. Gelatin has also fallen into disfavor due to concerns of mad cow disease.

Furthermore, soft capsule production using gelatin as a main component requires heating for a long time from dissolution to during production. As a result of heating, it is difficult to ensure a constant, reproducible quality for soft capsule production because the heating changes the structure of gelatin thus decreasing the viscosity of liquid, as well as similar structural changes.

Accordingly, some capsules using plant-derived materials have been developed.

JP-A-2003-504326 discloses a soft capsule using 1-carageenan and modified starch as vegetable starting materials. However, the use of starch and 1-carageenan in combination as starting materials requires a large amount of water for dissolving the starting materials. Therefore, drying of the capsule becomes a heavy burden, and the productivity is not entirely good.

JP-A-2003-55198 discloses a capsule based on a combination of water-soluble etherified starch as a vegetable starting material and a plasticizer. However, sufficient strength cannot be achieved during capsule formation due to insufficient of capsule shell sheet adhesiveness during capsule formation.

JP-A-2003-221323 discloses a soft capsule shell sheet containing a decomposition product of hydroxypropyletherified starch. However, even when combined with a plasticizer such as sorbitol, sufficient adhesiveness cannot be achieved. In addition, the capsule shell sheet lacks flexibility. Accordingly, capsule formation is not easy.

WO 03/049771 discloses a capsule shell sheet containing polyglutamic acid (PGA). However, preparation of a capsule containing PGA requires first drying a capsule shell sheet formed from a shell solution containing PGA over a long time. Accordingly, problems occur in that capsules cannot be produced continuously, caking occurs when a desiccant is not used during preservation due to the hygroscopicity of the capsule, and the sheet is generally insufficient.

Thus, there remains a critical need for the production of compositions for the production of capsule shells and capsule shells, per se, that possess sufficient strength and elongation as a capsule shell, superior adhesiveness and transparency, and a low burden on a drying step. The present invention provides a composition for a shell, which is suitable for producing a plant-derived, practical capsule shell sheet having both sufficient strength and elongation as a capsule shell, superior adhesiveness and transparency, and a low burden on a drying step, a capsule shell sheet obtained using the composition for a shell, and further a capsule preparation.

### SUMMARY OF THE INVENTION

It is an object of the present invention to provide a capsule shell that can be obtained by mixing (A) vegetable polysaccharide, (B) polyglutamic acid, and (C) water and, where necessary, at least one kind of material selected from (D) a plasticizer, (E) glycerin fatty acid ester and (F) oil.

That is, the present invention includes the following embodiments.

[1] A composition for a capsule shell, which comprises (A) a vegetable polysaccharide and (B) polyglutamic acid.

[2] The composition of the above-mentioned [1], which further comprises (C) water.

[3] The composition of the above-mentioned [1] or [2], which further comprises (D) a plasticizer.

[4] The composition of any of the above-mentioned [1] to [3], which further comprises (E) a glycerin fatty acid ester.

[5] The composition of any of the above-mentioned [1] to [4], which further comprises (F) an oil.

[6] The composition of any of the above-mentioned [1] to [5], wherein (A) is a starch.

[7] The composition of the above-mentioned [6], wherein the starch is selected from tapioca starch, corn starch, waxy corn starch, rice starch and wheat starch.

[8] The composition of any of the above-mentioned [1] to [7], wherein (A) is a modified vegetable polysaccharide.

[9] A capsule shell sheet, which comprises (A) a vegetable polysaccharide and (B) polyglutamic acid.

[10] A capsule preparation comprising (A) a vegetable polysaccharide and (B) polyglutamic acid.

The above objects highlight certain aspects of the invention. Additional objects, aspects and embodiments of the invention are found in the following detailed description of the invention.

### DETAILED DESCRIPTION OF THE INVENTION

Unless specifically defined, all technical and scientific terms used herein have the same meaning as commonly understood by a skilled artisan in the relevant art.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The composition for a capsule shell of the present invention contains at least one or more vegetable polysaccharides and a polyglutamic acid.

The kind of vegetable polysaccharides (component “(A)”) to be used in the capsule shell of the present invention is not particularly limited as long as it is a general starting material derived from a plant, and may be a natural product, a processed product and the like. Specifically, the vegetable polysaccharide may be a starch such as tapioca starch, corn starch, waxy corn starch, high-amylose corn starch, maize starch, potato starch, rice starch, wheat starch and the like; carageenan; agar; gellan gum; hydroxypropylmethylcellulose; sodium alginate; and the like. With respect to elongation and elasticity suitable for a capsule shell sheet, starch is preferable with tapioca starch, corn starch, waxy corn starch, rice starch and wheat starch the most preferable starches.

When a starch is selected as a main component of (A), a property to prevent sticking of capsules during a drying step can be imparted by adding a component other than the starch. The component other than the starch is not particularly limited. Nonetheless, carageenan, gellan gum, glucomannan and agar are preferred, with carageenan and gellan gum being more preferred, and carageenan the most preferred.

The component to be blended with the starch shows effect even at an amount of about 0.1-5 wt % of the total amount of component (A). When the amount is less than 0.1 wt %, a clear sticking-preventive effect may not be achieved sometimes, and when it exceeds 5 wt %, the transparency of the sheet may be influenced. To provide a useful effect, the amount of the blended component is preferably 0.2-3 wt %, particularly preferably 0.5-2 wt %, of the total amount of component (A).

Generally, vegetable polysaccharides (A) to be used for the composition for a capsule shell of the present invention may be modified or unmodified. As a specific modification method, a method based on hydroxypropyletherification, carboxymethylation and the like can be employed. From the aspect of elongation and hardness suitable for a capsule shell sheet, vegetable polysaccharides are preferable, vegetable polysaccharides modified by hydroxypropylation are more preferable, and modified vegetable polysaccharides decomposed by enzyme are more preferable.

When a modified vegetable polysaccharide is used as vegetable polysaccharide (A) to be used for the composition for a capsule shell of the present invention, the modified vegetable polysaccharide may be decomposed or not decomposed. As a specific method of decomposition, decomposition by heat, enzyme, acid and the like can be used. From the aspect of improvement in the elongation and elasticity suitable for a capsule shell sheet, a modified vegetable polysaccharide decomposed by enzyme is preferable.

The polyglutamic acid (component “(B)”) to be used for the composition for a capsule shell of the present invention is not particularly limited. Exemplary polyglutamic acids include: synthetic poly-α-glutamic acid, poly-γ-glutamic acid present in a viscous substance of natto or extrabacterially secreted by a member of the genus Bacillus, such as Bacillus natto and the like, a salt thereof and the like. Since these polyglutamic acids (B) can improve water solubility of vegetable polysaccharides, they can decrease the amount of water in the composition for a capsule shell. In addition, they improve adhesiveness of the capsule shell sheet as well as show a mineral solubilization effect in the lower section of the small intestine, thus promoting intestinal absorption of minerals. When used as a food material, poly-α-glutamic acid may be decomposed by protease during passage in the intestine, depending on the conditions of amount and the like. From the aspect of affording a high promoting effect on mineral absorption by the intestine, therefore, poly-γ-glutamic acid present in a viscous substance of natto or extrabacterially secreted by the Bacillus such as Bacillus natto and the like is preferable.

While the molecular weight of polyglutamic acid (B) to be used for the composition for a capsule shell of the present invention is not particularly limited, one having a weight average molecular weight of 3,000-1,000,000 is generally used. When it is less than 3,000, the adhesiveness improving effect on the capsule shell sheet is insufficient, and when it exceeds 1,000,000, dissolution and the like in a processing step become difficult. To maintain stable adhesion and the dissolution property of the starting materials, it is preferable that the polyglutamic acid have weight average molecular weight 10,000-100,000. The molecular weight is defined by a weight average molecular weight obtained using a gel permeation chromatography coupled to multi-angle laser light scattering method (GPC-MALLS method: Dawn DPS manufactured by Wyatt Technology).

The polyglutamic acid (B) to be used for the composition for a capsule shell of the present invention may be in a free form or may form a salt. The salt is not particularly limited and, for example, alkali metals such as sodium, potassium and the like, alkaline earth metals such as magnesium, calcium etc., and the like are used. From the aspect of versatility, sodium salt is preferable. As the kind of the salt, one kind may be used, or two or more kinds may be used in a mixture.

The amounts of (A) and (B) to be used for the composition for a capsule shell of the present invention can be appropriately determined depending on the kind of capsule and the kind of vegetable polysaccharides to be used, from the range where the capsule form and strength can be maintained. As the mixing ratio (mass ratio) of (A) and (B), the range of 99:1-30:70 is preferable. Particularly, in the case of a capsule using a starch shell, the mixing ratio (mass ratio) of (A) and (B) should be within the range of 99:1-60:40. When (A) exceeds 99:1, capsule forming becomes difficult because starch is hardly dissolved in water, resulting in an opaque sheet, and two capsule shell sheets are not adhered sufficiently by heat. On the other hand, when (B) exceeds 60:40, the strength of the capsule shell sheet becomes insufficient, and therefore, production of capsule becomes difficult. From the aspect of comprehensively securing the transparency, adhesiveness, strength and elongation necessary for a capsule shell sheet, the mixing ratio of (A) and (B) is preferably 95:5-60:40, more preferably 95:5-70:30, further preferably 95:5-80:20, and particularly preferably 90:10-80:20.

The composition for a capsule shell of the present invention contains water (component “(C)”). The water (C) to be used is not particularly limited as long as it is generally used for food. The component (C) is used to mainly dissolve component (A) and component (B), and to improve the capsule property. Here, the amount of water means a total amount of water contained in each component and water to be added during production of the composition. Examples of the water to be added include water used for dissolving or suspending each component as necessary, water used for dissolving or suspending the composition obtained by mixing each component, and the like.

The amount of water (C) contained in the composition for a capsule shell of the present invention is generally within the range of 20-55%, when calculated as a mass % to the total amount of those used as the capsule shell components from the components (A) to (F). When the amount is less than 20%, the solid contents may not be dissolved, and when it exceeds 55%, practical use may be difficult because of the heavy burden on a drying step. From the aspect of low burden on the capsule drying step, the upper limit of the water content is preferably 47%, more preferably 41%, and most preferably 35%. On the other hand, the lower limit of the water content is preferably 20%, more preferably 25%, and particularly preferably 30%, from the aspect of low burden on a drying step. When a step of high temperature high pressure treatment (dissolution treatment) using an extruder such as a real producing machine, the amount of (C) water to be added may shift toward 5 to 10% lower.

By further adding a plasticizer (component “(D)”) to the composition for a capsule shell of the present invention, a capsule shell sheet after film forming or a capsule can be softened. The plasticizer (D) is not particularly limited. Nonetheless, polyvalent alcohols such as glycerol, propylene glycol, 1,3-butyleneglycol and the like, sugar alcohols such as sorbitol, maltitol, erythritol, lactitol, xylitol and the like, saccharides such as glucose, fructose, sucrose and the like may be used. These may be used alone or two or more kinds thereof may be used in a mixture.

The weight % of (D) relative to the total weight of (A) and (B) can be within the range of 5-40%. When the weight is less than 5%, a capsule shell sheet may show degraded adhesiveness and also decreased transparency, and when the weight exceeds 40%, the sheet becomes too soft, sometimes resulting in poor strength and poor adhesiveness. Since the total evaluation of adhesiveness, transparency, elongation and strength becomes high, the lower limit is preferably 8%, more preferably 10%, and particularly preferably 12%. The upper limit is preferably 30%, more preferably 25%, further preferably 22%, and particularly preferably 18%.

By further adding a glycerin fatty acid ester (component “(E)”) in an amount within a certain range, the composition for a capsule shell of the present invention can enhance the coat strength of the capsule shell sheet.

The weight % of (E) relative to the total weight of (A) and (B) is within the range of 0.1-25%. When the weight is less than 0.1%, the strength of the capsule shell sheet may become low, and when it exceeds 25%, the strength of the capsule shell sheet may become low. From the aspect of production of a capsule shell sheet or capsule more superior in the strength, the lower limit is preferably 0.5%, more preferably 1%, further preferably 2%, still more preferably 4%, especially preferably 7%, and particularly preferably 10%. The upper limit is preferably 22%, more preferably 20%, further preferably 17%, and particularly preferably 15%.

By further adding an oil (component “(F)”) to the composition for capsule shell of the present invention, the detachability of the capsule shell sheet can be improved. (F) is not particularly limited, and soybean oil, canola oil, safflower oil, corn oil, coconut oil and palm oil can be specifically mentioned. These may be used alone or two or more kinds thereof may be used in a mixture. To improve detachability, corn oil, coconut oil and palm oil are preferable, coconut oil and palm oil are more preferable, and palm oil is particularly preferable.

The weight % of (F) relative to the total weight of (A) and (B) is within the range of 0.1-6%. When the weight is less than 0.1%, the detachability may be poor, and when the weight exceeds 6%, the strength may become low. Since a capsule shell sheet having strength and superior detachability can be obtained, the lower limit is preferably 0.2%, more preferably 0.4%, and particularly preferably 0.5%. The upper limit is preferably 5.0%, more preferably 3.0%, and particularly preferably 1.5%.

The capsule shell sheet of the present invention can contain other components generally used as shell components to the extent that does not inhibit the effect of the present invention. For example, additives known and in public use such as coloring agent, shielding agent, preservative, stabilizer, buffer and the like can be used, individually or in combination.

The capsule shell sheet of the present invention can be adapted to any form of various capsules such as hard capsule, soft capsule, seamless soft capsule and the like.

The shape, size and the like of capsule are not particularly limited. As for the form, round type, oval type, oblong type, tube type, teardrop type and the like can be produced. As for the size, a capsule of the size of several micrometers to several centimeters can be produced.

The composition for a shell of the present invention is processed to give a sheet for capsule with an extruder generally used for food processing, and then the capsule shell sheet is subjected to a generally-used soft capsule production machine according to the method described in JP-A-2003-70428 and the like. Specifically, firstly, a composition for a shell is treated at a high temperature and high pressure in an extruder, and the extruded material is formed into a capsule shell sheet with a thickness of about 0.8 mm using a die and a roll. The capsule shell sheet is fed, for example, into a rotary soft capsule production machine to give soft capsules. In the rotary soft capsule production machine, the capsule shell sheet is fed in between a pair of rotary cylindrical molds, and the content of the capsule is injected between the capsule shell sheets with a pump that works along therewith. At this time, the junction parts of the capsule shell sheet are heat-sealed by high pressure cut-off between convex teeth and teeth on the surface of a rotary cylindrical mold while heating to a suitable temperature to give a soft capsule.

Thereafter, the capsule is subjected to a drying step to dry to a desired water content of 10-12%, whereby a soft capsule can be obtained. In the case, the drying step is performed using a tumbler dryer and drying in shelf. In the case of a conventional capsule made only of starch, a large amount of water (about 50%) is required because it is not easily dissolved, and the drying time problematically becomes long. However, since the composition for capsule shell of the present invention can suppress the amount of water to be added, the drying time can be shortened and the productivity can be advantageously improved.

The capsule shell sheet of the present invention can also be made to contain the content components simultaneously with the capsule production in advance, whereby a capsule preparation can be formed. The content components may be other components generally used as the content of a capsule preparation. The form of the content components may be any of a liquid, a solid, and a suspension requiring particularly strong adhesiveness can also be encapsulated.

The capsule preparation of the present invention is widely useful in the field of pharmaceutical product, food, health food and the like as a practical capsule derived from a vegetable, which replaces conventional gelatin capsules.

While a composition for a capsule shell generally contains water as an essential component, the composition for a capsule shell of the present invention can reduce the water content of the composition for a capsule shell by a combined use of vegetable polysaccharides and polyglutamic acid. Therefore, the composition is advantageous in that the drying time can be shortened during formation of a shell sheet for the preparation of capsules, or during production of capsules, and the like. Since the shell sheet obtained from the composition is rich in flexibility and superior in tensile strength, capsules produced by a forming machine are not deformed, and capsules having a rugby ball shape like a gelatin capsule can be produced. Moreover, since capsule shell sheets show superior adhesiveness to each other, a suspension difficult to be filled in conventional vegetable capsules can be encapsulated. Shell sheets and capsule preparations obtained by processing the composition for a capsule shell of the present invention are entirely made of edible components and highly safe, and can be applied to various uses as biocompatible materials.

The capsule preparation of the present invention is widely useful in the fields of pharmaceutical product, food, health food and the like, as a practical plant-derived capsule replacing the existing gelatin capsules.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Materials and Methods—

The evaluation methods in the Examples are explained below.

Property Test of Capsule Shell Sheet:

A 1 mm-thick silicone sheet frame was placed on a 10 cm square PET film, each composition was flown in, and a 10 cm square PET film was placed thereon. This was sandwiched with steel plates and fixed with screws and heated for 5 min in an oil bath to 120° C. to give a capsule shell sheet. The obtained capsule shell sheet was evaluated for adhesiveness, film strength, elongation, and transparency.

Adhesiveness:

Two test pieces (1 cm×2 cm) were cut out from the capsule shell sheet obtained in the above, placed on one another and adhered with a heat sealer (Fuji Impulse Co., Ltd., P-200). After adhesion, the two pieces of capsule shell sheet were manually peeled off and the adhesiveness was evaluated. According to the dial setting of the heat sealer, the sheet was judged in 4 levels in Table 2. As used herein, adhesion means that the adhered surfaces of two pieces of sheets are melt fused to lose interface, and two pieces of sheets are not released on peeling.

TABLE 1

| evaluation item | evaluation |
|---|---|
| adhered at dial 6 | ⊚ |
| adhered at dial 7 | ◯ |
| adhered at dial 8 | Δ |
| not adhered at all at dials 6-8 | X |

In Table 1, the number of “dial” means heating for the time set. The length of time is dial 6<dial 7<dial 8.

Transparency:

The transparency of the capsule shell sheet was visually evaluated in 4 levels as shown in Table 2.

TABLE 2

| evaluation item | evaluation |
|---|---|
| completely transparent | ⊚ |
| somewhat transparent | ◯ |
| opaque | Δ |
| completely opaque | X |

Intensity and Elongation of Capsule Shell Sheet:

Film strength and elongation were evaluated using an all-purpose texture analyzer (SMS, TA.XT plus). The capsule shell sheet obtained above was set to a stage with a hole of diameter 150 mm. A rod with a ball (diameter 130 mm) on the tip was thrust into the surface of a capsule shell sheet at a rate of 0.5 cm/min, and the maximum strength and elongation at that time were measured.

As for the strength, the force applied on breakage was measured. The evaluation levels are as shown in Table 3.

TABLE 3

| evaluation range | evaluation |
|---|---|
| not less than 3.0 MPa | ⊚⊚ |
| not less than 2.8 MPa and less than 3.0 MPa | ◯⊚ |
| not less than 2.6 MPa and less than 2.8 MPa | ⊚ |
| not less than 2.4 MPa and less than 2.6 MPa | ◯ |
| not less than 2.2 MPa and less than 2.4 MPa | Δ |
| less than 2.2 MPa | X |

As for elongation, the length of elongation on breakage was measured. The evaluation levels are as shown in Table 4.

TABLE 4

| evaluation range | evaluation |
|---|---|
| not less than 10 mm | ⊚⊚ |
| not less than 9 mm and less than 10 mm | ◯⊚ |
| not less than 8 mm and less than 9 mm | ⊚ |
| not less than 7 mm and less than 8 mm | ◯ |
| not less than 6 mm and less than 7 mm | Δ |
| less than 6 mm | X |

Detachability:

In general, the surface of a capsule shell sheet comprising starch as a main starting material has considerably high stickiness as compared to a gelatin capsule shell sheet. Thus, continuous supply to a capsule forming machine is difficult. In addition, capsules produced from a highly sticky capsule shell sheet easily to each other. Therefore, production of a capsule shell sheet affording good detachability of the surface of a capsule shell sheet is important for continuous supply of a capsule shell sheet to a capsule forming machine.

The detachability was evaluated in 4 levels as shown in Table 5, based on the texture of a capsule shell sheet by sandwiching the sheet between fingers and releasing same.

TABLE 5

| evaluation item | evaluation |
|---|---|
| no sticking to finger | ⊚⊚ |
| sticky on fingers but immediately released by loosening hold | ◯⊚ |
| capsule shell sheet stretches between fingers and finally | ⊚ |
| released therefrom | |
| capsule shell sheet considerably stretches between fingers and | ◯ |
| finally released therefrom | |
| is not released from finger with ease | Δ |
| is not released from finger at all | X |

Evaluation of Burden on Drying Step:

A higher water content requires a longer time for a capsule drying step, and therefore, capsules were evaluated based on the criteria of Table 6.

TABLE 6

| amount of water added (%) | burden on drying step |
|---|---|
| -35 | ⊚ [symbol] (very light) |
| 36-41 | [symbol] (light) |
| 42-47 | [symbol] (average) |
| 48- | [symbol] (heavy) |

Each item except detachability was given the following points. The points were totaled and taken as the total evaluation score of a formed capsule.

TABLE 7

| item | evaluation score |
|---|---|
| ⊚⊚ | 5 |
| ⊚◯ | 4 |
| ⊚ | 3 |
| ◯ | 2 |
| Δ | 1 |
| X | 0 |

Starting Materials Used:

The materials to be used in Examples and Comparative Examples are explained in the following.
(A) hydroxypropyletherified starch (NIPPON STARCH CHEMICAL CO., LTD., G-800, water content 13%)
(B) γ-polyglutamic acid (Ajinomoto Co., Inc., food additive, molecular weight 28,000, water content 5%),
(C) Water includes water content of (A) and (B).
(D) glycerol (Lion Corporation, food additive, water content 0%)
(E) a glycerin fatty acid ester (Riken Vitamin Co., Ltd., G-002, water content 0%)
(F) palm oil (J-OIL MILLS. INC., product number Fry UP 201, water content 0%)

In Examples and Comparative Examples, the amount converted to a solid content is shown, and water is a total of the amount used for dissolving each solid content and added water.

### Examples 1-4

In Examples 1-4, adhesiveness, film strength, elongation and transparency at various ratios of (A) and (B) components were evaluated by the above-mentioned measurement methods. The results are as shown in Table 8.

TABLE 8

| Parameter | Ex. 1 | Ex. 2 | Ex. 3 | Ex. 4 |
|---|---|---|---|---|
| (A) | 54% | 51% | 48% | 42% |
| (B) | 6% | 9% | 12% | 18% |
| (C) | 40% | 40% | 40% | 40% |
| total | 100% | 100% | 100% | 100% |
| (A)/(A) + (B) | 90% | 85% | 80% | 70% |
| (B)/(A) + (B) | 10% | 15% | 20% | 30% |
| adhesiveness | ◯ | ⊚ | ⊚ | ◯ |
| transparency | ◯ | ◯ | ◯ | ◯ |
| strength | ⊚ | ⊚ | ◯ | Δ |
| elongation | ◯ | ◯ | ◯ | ◯ |
| burden on drying step | ⊚ | ⊚ | ⊚ | ⊚ |
| total evaluation | 12 | 13 | 12 | 10 |

### Examples 5-8 and Comparative Examples 1-2

In Examples 5-8, adhesiveness, film strength, elongation and transparency were evaluated at various ratios of (A) and (B) components and with addition of component (D), according to the above-mentioned measurement method. In Comparative Examples 1-2, adhesiveness, film strength, elongation and transparency were evaluated without component (A) or (B), according to the above-mentioned measurement method. The results are as shown in Table 9.

TABLE 9

| | | | | | Com. | Com. |
|---|---|---|---|---|---|---|
| Parameter | Ex. 5 | Ex. 6 | Ex. 7 | Ex. 8 | Ex. 1 | Ex. 2 |
| (A) | 46% | 42% | 37% | 32% | 51% | 0% |
| (B) | 5% | 10% | 15% | 20% | 0% | 55% |
| (C) | 41% | 41% | 40% | 40% | 42% | 37% |
| (D) | 8% | 8% | 8% | 8% | 8% | 8% |
| total | 100% | 100% | 100% | 100% | 100% | 100% |
| (A)/(A) + (B) | 90.2% | 80.8% | 71.2% | 61.5% | 100.0% | 0.0% |
| (B)/(A) + (B) | 9.8% | 19.2% | 28.8% | 38.5% | 0.0% | 100.0% |
| adhesiveness | ◯ | ⊚ | ◯ | ◯ | X | — |
| transparency | ◯ | ⊚ | ◯ | ⊚ | X | — |
| strength | ⊚ | ⊚ | ⊚ | Δ | ◯ | — |
| elongation | ◯ | ⊚ | ◯ | ⊚ | ◯ | — |
| burden on | ⊚ | ⊚ | ⊚ | ⊚ | ◯ | ◯ |
| drying step | | | | | | |
| total | 12 | 15 | 12 | 12 | 6 | 2 |
| evaluation | | | | | | |

— means measurement was not possible due to liquid.

### Examples 9-14 and Comparative Examples 3-6

In Examples 9-14, capsule shell sheets were prepared in the same manner as in Examples 1-4 and at various amount of water added from 33% to 47%, and evaluated for the adhesiveness, film strength, elongation and transparency. In Comparative Examples 3-6, capsule shell sheets were prepared in the same manner as in Examples 1-4 using a composition free of PGA and evaluated for the adhesiveness, film strength, elongation and transparency. The results are as shown in Table 10.

TABLE 10-1

| Parameter | Ex. 9 | Ex. 10 | Ex. 11 | Ex. 12 | Ex. 13 | Ex. 14 |
|---|---|---|---|---|---|---|
| (A) | 48% | 46% | 43% | 42% | 40% | 37% |
| (B) | 11% | 11% | 11% | 10% | 10% | 9% |
| (C) | 33% | 35% | 38% | 41% | 43% | 47% |
| (D) | 8% | 8% | 8% | 7% | 7% | 7% |
| total | 100% | 100% | 100% | 100% | 100% | 100% |
| (A)/(A) + (B) | 81.3% | 80.7% | 79.6% | 80.8% | 80.0% | 80.4% |
| (B)/(A) + (B) | 18.6% | 19.3% | 20.4% | 19.2% | 20.0% | 19.6% |
| adhesiveness | Δ | ◯ | ◯ | ⊚ | ◯ | ◯ |
| transparency | ◯ | ◯ | ⊚ | ⊚ | ⊚ | ⊚ |
| strength | ⊚ | ⊚ | ⊚ | ⊚ | ◯ | Δ |
| elongation | ◯ | ◯ | ⊚ | ⊚ | ⊚ | ⊚ |
| burden on | ⊚◯ | ⊚◯ | ⊚ | ⊚ | ◯ | ◯ |
| drying step | | | | | | |
| total | 12 | 13 | 14 | 15 | 12 | 11 |
| evaluation | | | | | | |

TABLE 10-2

| | Comparative | Comparative | Comparative | Comparative |
|---|---|---|---|---|
| Parameter | Example 3 | Example 4 | Example 5 | Example 6 |
| (A) | 51% | 49% | 45% | 44% |
| (B) | 0% | 0% | 0% | 0% |
| (C) | 7% | 7% | 7% | 6% |
| (D) | 42% | 44% | 48% | 50% |
| total | 100% | 100% | 100% | 100% |
| (A)/(A) + (B) | 100% | 100% | 100% | 100% |
| (B)/(A) + (B) | 0% | 0% | 0% | 0% |
| adhesiveness | — | Δ | ◯ | Δ |
| transparency | — | X | Δ | ◯ |
| strength | — | ◯ | Δ | Δ |
| elongation | — | X | Δ | ◯ |
| burden on | ◯ | ◯ | X | X |
| drying step | | | | |
| total | 2 | 5 | 5 | 6 |
| evaluation | | | | |

— means sheet could not be produced because solid content was not dissolved in water.

### Examples 15-21

In Examples 15-21, component (D) was increased, component (C) was reduced, and adhesiveness, film strength, elongation and transparency were evaluated by the above-mentioned measurement methods. The results are as shown in Table 11.

TABLE 11

| Parameter | Ex. 15 | Ex. 16 | Ex. 17 | Ex. 18 | Ex. 19 | Ex. 20 | Ex. 21 |
|---|---|---|---|---|---|---|---|
| (A) | 45% | 44% | 42% | 39% | 36% | 39% | 40% |
| (B) | 11% | 10% | 10% | 9% | 9% | 10% | 10% |
| (C) | 41% | 41% | 41% | 40% | 40% | 34% | 32% |
| (D) | 4% | 5% | 8% | 12% | 15% | 17% | 18% |
| total | 100% | 100% | 100% | 100% | 100% | 100% | 100% |
| (B)/(A) + (B) | 19.6% | 18.5% | 19.2% | 18.8% | 20.0% | 19.4% | 20.0% |
| (D)/(A) + (B) | 7.1% | 9.3% | 15.4% | 25.0% | 33.3% | 34.1% | 36.0% |
| adhesiveness | ◯ | ⊚ | ⊚ | ⊚ | ◯ | ⊚ | ⊚ |
| transparency | Δ | ◯ | ⊚ | ⊚ | ⊚ | ⊚ | ⊚ |
| strength | ⊚ | ⊚ | ⊚ | ◯ | Δ | ⊚ | ◯ |
| elongation | ◯ | ◯ | ⊚ | ⊚ | ⊚ | ⊚ | ⊚ |
| burden on | ⊚ | ⊚ | ⊚ | ⊚ | ⊚ | ⊚◯ | ⊚◯ |
| drying step | | | | | | | |
| total | 11 | 13 | 15 | 14 | 12 | 16 | 15 |
| evaluation | | | | | | | |

### Examples 22-26

In Examples 22-26, capsule shell sheets were prepared in the same manner as in Examples 1-4 with various amounts of (E) glycerin fatty acid ester, and adhesiveness, film strength, elongation and transparency were evaluated. The results are as shown in Table 12.

TABLE 12

| Parameter | Ex. 22 | Ex. 23 | Ex. 24 | Ex. 25 | Ex. 26 |
|---|---|---|---|---|---|
| (A) | 41% | 40% | 37% | 36% | 35% |
| (B) | 10% | 10% | 9% | 9% | 8% |
| (C) | 41% | 41% | 41% | 41% | 41% |
| (D) | 8% | 7% | 7% | 7% | 6% |
| (E) | 1% | 3% | 6% | 8% | 10% |
| total | 100% | 100% | 100% | 100% | 100% |
| (B)/(A) + (B) | 19.6% | 20.0% | 19.6% | 20.0% | 18.6% |
| (D)/(A) + (B) | 15.7% | 14.0% | 15.2% | 15.6% | 14.0% |
| (E)/(A) + (B) | 2.0% | 6.0% | 13.0% | 17.8% | 23.3% |
| adhesiveness | ⊚ | ⊚ | ⊚ | ⊚ | ⊚ |
| transparency | ⊚ | ⊚ | ⊚ | ⊚ | ⊚ |
| strength | ⊚ | ◯⊚ | ⊚⊚ | ⊚ | ◯ |
| elongation | ⊚ | ⊚ | ◯⊚ | ◯⊚ | ◯⊚ |
| burden on | ⊚ | ⊚ | ⊚ | ⊚ | ⊚ |
| drying step | | | | | |
| total | 15 | 16 | 18 | 16 | 15 |
| evaluation | | | | | |

### Examples 27-30

In Examples 27-30, the evaluation was performed with various ratios of oil. Oil was added and the property was measured. The results are as shown in Table 13.

TABLE 13

| Parameter | Ex. 27 | Ex. 28 | Ex. 29 | Ex. 30 |
|---|---|---|---|---|
| (A) | 37% | 37% | 37% | 36% |
| (B) | 9% | 9% | 9% | 9% |
| (C) | 41% | 41% | 42% | 43% |
| (D) | 7% | 7% | 7% | 7% |
| (E) | 6% | 6% | 6% | 6% |
| (F) | 0.25% | 0.50% | 1.0% | 2.0% |
| total | 100% | 100% | 100% | 100% |
| (B)/(A) + (B) | 19.6% | 19.6% | 19.6% | 20.0% |
| (D)/(A) + (B) | 15.2% | 15.2% | 15.2% | 15.6% |
| (E)/(A) + (B) | 13.0% | 13.0% | 13.0% | 13.3% |
| (F)/(A) + (B) | 0.5% | 1.1% | 2.2% | 4.4% |
| adhesiveness | ⊚ | ⊚ | ⊚ | ⊚ |
| transparency | ⊚ | ⊚ | ⊚ | ⊚ |
| strength | ◯⊚ | ⊚⊚ | ◯ | Δ |
| elongation | ◯⊚ | ⊚⊚ | ⊚ | ◯ |
| burden on drying step | ⊚ | ⊚ | ◯ | ◯ |
| total evaluation | 17 | 19 | 13 | 11 |
| detachability | ⊚ | ⊚⊚ | ⊚⊚ | ⊚⊚ |

### Production Examples

Production Examples performed in the state close to the actual production are shown below. The evaluation methods are first explained.

Elongation and Elastic Modulus:

A test piece (6 mm×60 mm) was prepared from a sheet obtained from a conical die. The test piece was set to TENSILON (ORIENTEC, RTC-1250A) and elongation (ratio of elongated part relative to original sheet) and elastic modulus (MPa) were measured at a tensile rate of 50 mm/min.

In addition, the elongation and elastic modulus of a sheet suitable for capsulation were defined in three levels as follows.

1. Elongation
◯ . . . not less than 80%
Δ . . . 79-40%
× . . . not more than 39%

2. Elastic modulus
◯ . . . not more than 0.39 MPa
Δ . . . 0.40-0.79 MPa
× . . . not less than 0.80 MPa
   Adhesiveness:

Two pieces of sheets were applied to a capsule forming machine. When the sheets were adhered to each other, the bonded surface was free of concaves and convexes, and the content filled therein did not leak out after contact with a hand, the capsule was evaluated to “have adhesiveness”. When the content could be filled in but the bonded surface had concaves and convexes, and the content leaked out after contact with a hand, the capsule was evaluated to have “no adhesiveness”.

Stamping:

To produce a capsule by stamping, a capsule shell needs to have sufficient strength and detachability from a mold. When a capsule could be formed by stamping two pieces of sheets using the mold of a capsule forming machine, the sheet was evaluated as “good” and a sheet failed to provide a capsule by stamping (indetachable from the sheet) was evaluated as “no good”.

State after Drying:

The hardness and elasticity of a capsule after drying (water content of capsule shell 10-12%) were evaluated by the feel of hand.

Transparency:

The transparency of capsule was visually evaluated into “transparent” and “semitransparent”.

Stickiness of Capsule after Forming>

The stickiness was evaluated in 3 levels shown in the following Table 14 by the feel of hand.

TABLE 14

| | |
|---|---|
| no stickiness at all | ⊚ |
| almost no stickiness | ◯ |
| Sticky | X |

### Production Example 1

The starting materials were mixed according to the following formulation and were applied to an extruder (real machine) and treated at a high pressure. Using a conical die set at the extruder outlet, a 1 mm thick sheet was prepared.

The operation conditions of an extruder (2 axis extruder EA-20 manufactured by Suehiro EPM Corporation) were rotation: 80 rpm, pressure: 2.0 kg/cm², carrier part temperature: 90° C., temperature of temperature rise part: 110° C. However, only in Production Example 1, Production Comparative Examples 1 and 2, the real amounts used of the shell materials fed into the extruder are indicated. In “( . . . )”, a breakdown of the net solid amount and the amount of water is indicated. As for the relationship in amount of each component of a composition for a capsule shell, calculated values are indicated hereunder.

| water added | 30.8 | wt % | |
|---|---|---|---|
| hydroxypropyletherified starch (G- | 46.2 | wt % | (net solid amount |
| 800, manufactured by NIPPON | | | 40.2 wt % + |
| STARCH CHEMICAL CO., LTD.) | | | water 6.0 wt %) |
| hydroxypropyletherified starch | 7.4 | wt % | (net solid amount |
| decomposed product (PKW, | | | 6.4 wt % + |
| manufactured by NIPPON STARCH | | | water 1.0 wt %) |
| CHEMICAL CO., LTD.) | | | |
| γ-polyglutamic acid (food additive, | 7.4 | wt % | (net solid amount |
| manufactured by Ajinomoto Co., | | | 7.0 wt % + |
| Inc., molecular weight 28,000) | | | water 0.4 wt %) |
| glycerol (food additive, | 7.4 | wt % | |
| manufactured by Lion Corporation) | | | |
| propylene glycol (food additive, | 0.3 | wt % | |
| manufactured by JUNSEI | | | |
| CHEMICAL CO., LTD.) | | | |
| glycerin fatty acid ester (G-002, | 0.5 | wt % | |
| manufactured by Riken Vitamin Co., | | | |
| Ltd.) | | | |
| (A)/(A) + (B) | 86.9% | | |
| (B)/(A) + (B) | 13.1% | | |
| total amount of component (C) | 38.1 | wt % | |

| | |
|---|---|
| (D)/(A) + (B) | 13.8% |

(E)/(A)+(B) 0.9%

### Production Example 2

A sheet was prepared in the same manner as in Production Example 1 except that the material composition alone was changed.

Weight composition of shell materials fed into the extruder:

| water added | 23.1 | wt % | |
|---|---|---|---|
| hydroxypropyletherified starch (G- | 53.8 | wt % | (net solid amount |
| 800, manufactured by NIPPON | | | 46.8 wt % + |
| STARCH CHEMICAL CO., LTD.) | | | water 7.0 wt %) |
| κ-carageenan (WR-80-J, | 0.8 | wt % | (net solid amount |
| manufactured by Sansho Co., Ltd.) | | | 0.7 wt % + |
| | | | water 0.1 wt %) |
| γ-polyglutamic acid (food additive, | 7.7 | wt % | (net solid amount |
| manufactured by Ajinomoto Co., Inc., | | | 7.3 wt % + |
| molecular weight 28,000) | | | water 0.4 wt %) |
| glycerol (food additive, manufactured | 13.8 | wt % | |
| by Lion Corporation) | | | |
| glycerin fatty acid ester (G-002, | 0.8 | wt % | |
| manufactured by Riken Vitamin Co., | | | |
| Ltd.) | | | |
| (A)/(A) + (B) | 86.7% | | |
| (B)/(A) + (B) | 13.3% | | |
| total amount of component (C) | 30.6 | wt % | |

| | |
|---|---|
| (D)/(A) + (B) | 25.2% |
| (E)/(A) + (B) | 1.5% |
| (κ-carageenan)/(A) | 1.5% |

### Production Comparative Example 1

A sheet was prepared in the same manner as in Production Example 1 except that the material composition alone was changed. The motor load was 10A.

Weight composition of shell materials fed into the extruder (amount of water does not include the amount of water contained in each material).

| water added | 30.8 | wt % | |
|---|---|---|---|
| hydroxypropyletherified starch (G- | 59.9 | wt % | (net solid amount |
| 800, manufactured by NIPPON | | | 52.1 wt % + |
| STARCH CHEMICAL CO., LTD.) | | | water 7.8 wt %) |
| hydroxypropyletherified starch | 3.0 | wt % | (net solid amount |
| decomposed product (PKW, | | | 2.6 wt % + |
| manufactured by NIPPON STARCH | | | water 0.4 wt %) |
| CHEMICAL CO., LTD.) | | | |
| glycerol (food additive, | 6.0 | wt % | |
| manufactured by Lion Corporation) | | | |
| propylene glycol (food additive, | 0.3 | wt % | |
| manufactured by JUNSEI | | | |
| CHEMICAL CO., LTD.) | | | |
| (A)/(A) + (B) | 100.0% | | |
| (B)/(A) + (B) | 0.0% | | |
| total amount of component (C) | 39.0 | wt % | |

| | |
|---|---|
| (D)/(A) + (B) | 11.0% |
| (E)/(A) + (B) | 0.5% |

### Production Comparative Example 2

A sheet was prepared in the same manner as in Production Example 1 except that the material composition alone was changed. The motor load was 5.2A. The amount of water including the amount of water contained in the powder was 40% relative to the total amount.

Weight composition of shell materials fed into the extruder:

| water added | 30.8 | wt % | |
|---|---|---|---|
| hydroxypropyletherified starch (G- | 49.9 | wt % | (net solid amount |
| 800, manufactured by NIPPON | | | 43.4 wt % + |
| STARCH CHEMICAL CO., LTD.) | | | water 6.5 wt %) |
| hydroxypropyletherified starch | 11.1 | wt % | (net solid amount |
| decomposed product (PKW, | | | 9.7 wt % + |
| manufactured by NIPPON STARCH | | | water 1.4 wt %) |
| CHEMICAL CO., LTD.) | | | |
| glycerol (food additive, | 7.4 | wt % | |
| manufactured by Lion Corporation) | | | |
| propylene glycol (food additive, | 0.3 | wt % | |
| manufactured by JUNSEI | | | |
| CHEMICAL CO., LTD.) | | | |
| glycerin fatty acid ester (G-002, | 0.5 | wt % | |
| manufactured by Riken Vitamin Co., | | | |
| Ltd.) | | | |
| (A)/(A) + (B) | 100.0% | | |
| (B)/(A) + (B) | 0.0% | | |
| total amount of component (C) | 38.7 | wt % | |

| | |
|---|---|
| (D)/(A) + (B) | 13.9% |
| (E)/(A) + (B) | 0.9% |

As Production Example 1 (containing polyglutamic acid), Production Example 2 (containing polyglutamic acid, κ-carageenan), Production Comparative Examples 1, 2 (without polyglutamic acid), soft capsules were prepared by a continuous rotary die capsule filling machine using shell sheets prepared as mentioned above and a capsule content (medium chain fatty acid), and evaluated. The results are shown in Table 15.

TABLE 15
Property evaluation of capsule shell composition

| | | | Production | Production |
|---|---|---|---|---|
| | Production | Production | Comparative | Comparative |
| Parameter | Example 1 | Example 2 | Example 1 | Example 2 |
| elongation (%) | ◯(81) | ◯(95) | X(38) | Δ(62) |
| elastic modulus | ◯ | ◯ | X | X |
| (MPa) | (0.33) | (0.27) | (1.80) | (1.90) |
| transparency | transparent | transparent | semi- | semi- |
| | | | transparent | transparent |
| adhesiveness | yes | yes | none | none |
| stamping | good | good | no good | no good |
| (strength, | | | | |
| detachability) | | | | |
| stickiness of | ◯ | ⊚ | ◯ | ◯ |
| capsule | | | | |
| after forming | | | | |
| state after | soft | soft | stiff | stiff |
| drying | | | | |

As shown in Table 15, without addition of polyglutamic acid, elongation and elastic modulus become insufficient, the transparency, adhesiveness and stamping performance become poor, and only a capsule stiff after drying can be obtained (Production Comparative Examples 1, 2).

However, it has been clarified that, by the addition of polyglutamic acid, the elongation of a sheet can be improved, the elastic modulus becomes small, a sheet more suitable for capsule forming can be produced, and the addition is effective for adhesiveness between sheets made of the shell composition, as well as the softness of capsule after drying (Production Example 1).

Moreover, it has also been clarified that the elongation of a shell composition can be improved, a capsule can be produced by stamping using a mold of a capsule forming machine, and the transparency can also be improved. In addition, by the addition of κ-carageenan by 1.5% as component A, the stickiness immediately after forming can be prevented (Production Example 2).

Numerous modifications and variations on the present invention are possible in light of the above teachings. It is, therefore, to be understood that within the scope of the accompanying claims, the invention may be practiced otherwise than as specifically described herein.

1. A capsule shell composition, which comprises (A) a vegetable polysaccharide and (B) polyglutamic acid.

2. The composition of claim 1, wherein (A) is a vegetable polysaccharide obtained from a plant, natural product, or processed product and is selected from the group consisting of a starch, carageenan, agar, gellan gum, hydroxypropylmethylcellulose, and sodium alginate.

3. The composition of claim 1, wherein (A) is a starch.

4. The composition of claim 3, wherein the starch is selected from the group consisting of tapioca starch, corn starch, waxy corn starch, rice starch, and wheat starch.

5. The composition of claim 1, wherein (B) is selected from the group consisting of a synthetic poly-α-glutamic acid, a poly-γ-glutamic acid present in a viscous substance of natto and a extrabacterially secreted poly-α-glutamic acid by a member of the genus Bacillus.

6. The composition of claim 1, wherein (B) is a polyglutamic acid having a weight average molecular weight ranging from 3,000 to 1,000,000.

7. The composition of claim 1, wherein the (A) and (B) are in a mass ratio of 99:1 to 30:70.

8. The composition of claim 1, wherein said composition comprises at least one additional component selected from the group consisting of:
(C) water
(D) one or more plasticizers;
(E) one or more glycerin fatty acid esters; and
(F) one or more oils.

9. The composition of claim 1, further comprising (C) water.

10. The composition of claim 9, wherein said water is at an amount ranging from 20 to 55%.

11. The composition of claim 9, further comprising (D) one or more plasticizers.

12. The composition of claim 11, wherein said plasticizers are selected from the group consisting of a polyvalent alcohol, a sugar alcohol, and a saccharide.

13. The composition of claim 11, wherein the weight % of (D) relative to the total weight of (A) and (B) is within the range of 5 to 40%.

14. The composition of claim 11, further comprising (E) one or more glycerin fatty acid esters.

15. The composition of claim 14, wherein the weight % of (E) relative to the total weight of (A) and (B) is within the range of 0.1 to 25%.

16. The composition of claim 14, further comprising (F) one or more oils.

17. The composition of claim 16, wherein said oils are selected from the group consisting of soybean oil, canola oil, safflower oil, corn oil, coconut oil, and palm oil.

18. The composition of claim 16, wherein the weight % of (F) relative to the total weight of (A) and (B) is within the range of 0.1 to 6%.

19. A capsule shell sheet, which comprises the composition of claim 1.

20. A method of making a capsule shell comprising using the composition of claim 1 as the starting material for the capsule shell to reduce water content.
